(19) United States
(12) Patent Application Publication
(10) Pub. No.: US 2025/0114098 A1
(43) Pub. Date: Apr. 10, 2025

(54) Coronary Sinus Occlusion Systems, Devices and Methods

(71) Applicant: VahatiCor, Inc., Los Gatos, CA (US)

(72) Inventors: Kevin H. Van Bladel, Livermore, CA (US); Benjamin M. Trapp, Scottsdale, AZ (US); Marwan Berrada-Sounni, Santa Clara, CA (US)

(21) Appl. No.: 18/833,179

(22) PCT Filed: Feb. 6, 2023
(86) PCT No.: PCT/US2023/012374

§ 371 Date: Jul. 25, 2024

Related U.S. Application Data
(60) Provisional application No. 63/306,581, filed on Feb. 4, 2022.

(51) Int. Cl. A61B 17/12; A61B 17/00; A61M 25/10

(52) U.S. Cl. CPC A61B 17/12109; A61B 17/1204; A61B 17/12136; A61B 2017/00022; A61M 2025/1052

(57) ABSTRACT

Systems, devices and methods of adjustable and controlled vascular occlusion of the coronary sinus and other vessels using catheter-mounted and implantable occlusion devices that facilitate retrograde drug infusion and pressure monitoring are disclosed. Disclosed systems, devices and methods include balloons and self-supporting adjustable flow restricting structures for improved retrograde perfusion after a myocardial infarct.

### RELATED APPLICATION DATA

This application claims the benefit of priority of U.S. Provisional Patent Application Ser. No. 63/306,581, filed Feb. 4, 2022, and titled “Coronary Sinus Occlusion System”, which is incorporated by reference herein in its entirety.

### FIELD OF THE DISCLOSURE

The present disclosure generally relates to the field of vascular and cardiac occlusion devices to increase venous pressure and provide redistribution of blood flow, as well as provide retrograde perfusion after a myocardial infarct. In particular, the present disclosure is directed to systems, devices and methods of adjustable and controlled vascular occlusion device with retrograde drug infusion.

### BACKGROUND

Occlusion of the coronary sinus has been studied for controlling the flow within the vasculature after ST-segment elevation myocardial infarction (STEMI). Studies in patients of intermittent coronary sinus occlusion following a STEMI has shown promise in reduced infarct size attributed to re-distribution of the flow to the deprived perfusion border zones and increased flow. The intermittent occlusion has been described by Mohl in U.S. Pat. No. 8,500,686 to Miracor Medical. This prior art describes a balloon catheter with a pressure-controlled intermittent coronary sinus occlusion (PICSCO) treatment to the heart. The catheter must remain within the patient for two hours as the pressure is monitored, and the balloon is inflated periodically to occlude the coronary sinus to improve retrograde perfusion of the vasculature. In another prior art example, Ben-Muvhar et al. in U.S. Pat. No. 8,858,612 describes a flow-reducing implant in the coronary sinus for refractory angina pain. However, it is specifically not configured to completely occlude the vessel.

While the above prior art presents some solutions in certain applications, there remains a need in the art for improved devices with broader application.

### SUMMARY OF THE DISCLOSURE

To address ongoing needs in the art, the present disclosure describes systems, devices and methods for infusing therapeutic agents within the coronary sinus to improve/optimize myocardial salvage and reperfusion to limit the infarct size while a vessel is occluded. In addition, the medications can be injected into the middle cardiac vein that would enable the inferior wall (RCA STEMI patients) to be treated in addition to LAD and LCx STEMIs. Systems, devices and methods are also disclosed providing a fully implantable, intermittent occlusion of the coronary sinus.

In some embodiments, vascular pressure monitoring systems comprise a catheter body defining at least a guidewire lumen and an inflation lumen and at least one balloon communicating with the inflation lumen, distally disposed on the catheter body to define a vascular flow restriction when inflated within a vascular lumen. Pressure sensors may include a distal pressure sensor disposed distally with respect to the balloon and a proximal pressure sensor disposed proximally with respect to the balloon. The at least one balloon may be configured to provide a variable flow restriction. Optionally a guide catheter is provided and configured to receive the catheter body. A second proximal pressure sensor may be disposed on the guide catheter.

In one alternative embodiment, the at least one balloon comprises an inner, non-compliant balloon and an outer compliant balloon. The catheter body comprises a triple lumen catheter with a first inflation lumen communicating with the inner, non-compliant balloon, a second inflation lumen communicating with the outer compliant balloon and a guidewire lumen. The inner, non-compliant balloon when inflated provides a minimum fixed vascular flow restriction and the outer compliant balloon may be further variably inflated to provide a greater variable flow restriction.

In another alternative embodiment, the at least one balloon comprises an hour-glass shaped balloon defining an internal blood flow orifice, with the internal orifice defined by an inner, annular compliant balloon wall to provide a variable flow restriction.

In a further alternative embodiment, the at least one balloon is disposed around the catheter body and the catheter body defines a flow passage through the balloon having an open distal end defining one blood flow port and a side hole proximal to the balloon defining a second blood flow port. A flow control stylus may be disposed in the flow passage and moved longitudinally with respect to the side hole to provide a variable flow restriction.

In other embodiments, implantable variable vascular flow restriction devices comprise an implantable body member configured to be implanted in a vascular lumen and to extend across the vascular lumen fully around the inner periphery of the vascular lumen with variable flow restricting means disposed within the implantable body member, and means for controlling the variable flow restricting means also provided.

In one alternative embodiment, the variable flow restricting means comprises a pressure sensor configured to produce a local vascular pressure signal and an adjustable diameter orifice responsive to said pressure signal. Means for controlling may also comprise a vibratory energy source cooperating with the variable flow restricting means to set the adjustable orifice diameter in response to the pressure signal. Alternatively, the means for controlling may comprise a remotely deliverable heating source responsive to said pressure signal and a shape-memory alloy having a temperature variable dimensional property forming said adjustable diameter orifice, whereby delivery of heat alters the orifice diameter and removal of heat returns the orifice diameter to an original size.

In another alternative embodiment, the variable flow restricting means comprises a fixed plate defining a first elongate opening and a rotatable plate defining a second elongate opening wherein the second plate overlies the first plate and rotation of the second plate relative to the first plate aligns the elongate openings to provide a variable area flow passage.

Other embodiments of the present disclosure relate to a vascular diagnostic or treatment method, comprising placing a vascular pressure monitoring catheter into a vessel lumen at treatment or monitoring site, inflating a distal balloon on the catheter at the treatment or monitoring site to at least partially occlude the vessel lumen, delivering a therapeutic or diagnostic agent into the vessel lumen through the catheter beyond the partial occlusion, and monitoring patient response to the therapeutic or diagnostic agent.

### BRIEF DESCRIPTION OF THE DRAWINGS

For the purpose of illustrating the disclosure, the drawings show aspects of one or more embodiments of the disclosure. However, it should be understood that the present disclosure is not limited to the precise arrangements and instrumentalities shown in the drawings, wherein:

FIG. 1 schematically depicts a vascular pressure monitoring system according to the present disclosure disposed in the coronary sinus.

FIG. 2 is a cross-sectional view through line A-A of the vascular pressure monitoring system shown in FIG. 1.

FIG. 3 schematically depicts a proximal hub arrangement for a vascular pressure monitoring system according to the present disclosure.

FIG. 4 schematically depicts an alternative embodiment of a vascular pressure monitoring system according to the present disclosure.

FIG. 5 is a cross-sectional view through line B-B of the vascular pressure monitoring system shown in FIG. 4.

FIG. 6 is a schematic cross-section of a further alternative embodiment of an occlusion catheter with a stylet.

FIG. 7 is another schematic cross-section of the embodiment shown in FIG. 6 with the stylet pulled back to allow blood flow through the catheter.

FIG. 8 is a schematic depiction of the heart and coronary vessels showing infusing therapeutic agents retrograde into the vascular system.

FIG. 9 is a schematic depiction of the heart and coronary vessels showing access to the middle cardiac vein for retrograde perfusion.

FIG. 10 is a schematic depiction of the heart and coronary vessels showing distal perfusion to obtain a controlled angiogram within the vascular system.

FIG. 11 is a schematic depiction of the heart and coronary vessels showing an implant that can be positioned within the coronary sinus without being tethered from outside the body.

FIGS. 12A-C schematically depict different occlusion levels for an embodiment of an implant that can be cycled to cause an occlusion of the vessel with an external coupling, wherein FIG. 12A depicts a closed state, FIG. 12B depicts a restrictive state and FIG. 12C depicts an open state.

FIG. 13 schematically shows a coupling to an external source that alternates the restrictive flow through periods of no restriction and less restriction.

FIG. 14 is a block diagram schematically depicting an embodiment of a disclosed occlusion device with RF communication and ability to control the cycling of the implant with acoustic energy transmission.

FIGS. 15A-C schematically depict an embodiment of a device with an orifice that changes diameter with a pressure sensor and actuator, wherein FIG. 15A depicts a closed state, FIG. 15B depicts a restrictive state and FIG. 15C depicts an open state.

FIGS. 16A, 16B, 16C, 16D, and 16E schematically depict different states of occlusion for an embodiment of a device employing a turbine with two apertures/plates where one of the plates spin to alternate the hole alignment allowing occlusion or flow as it is rotated.

FIGS. 17A, 17B, 17C and 17D schematically depict a further alternative embodiment of an occlusion device employing aperture rotation using electromagnetic energy.

FIGS. 18A, 18B, 18C, 18D, and 18E schematically depict another alternative embodiment of an occlusion device employing aperture rotation using acoustic ultrasound with directional vibration to rotate fan blades to open and close the apertures.

FIGS. 19A, 19B, 19C and 19D schematically depict yet a further alternative embodiment of an occlusion device employing a cone/piston that obstructs an orifice that can be translated back and forth using linear electromagnetic energy.

### DETAILED DESCRIPTION

To assist in monitoring pressure and occluding the coronary sinus pressure, embodiments disclosed provide a temporary occlusion, which may be partial occlusion or full occlusion, to provide inter alia hemodynamic response of retrograde perfusion back into the coronary arteries. Occlusion or restriction of the coronary sinus may increase venous pressure and provide redistribution of blood flow at the capillary level, potentially perfusing areas that were otherwise ischemic.

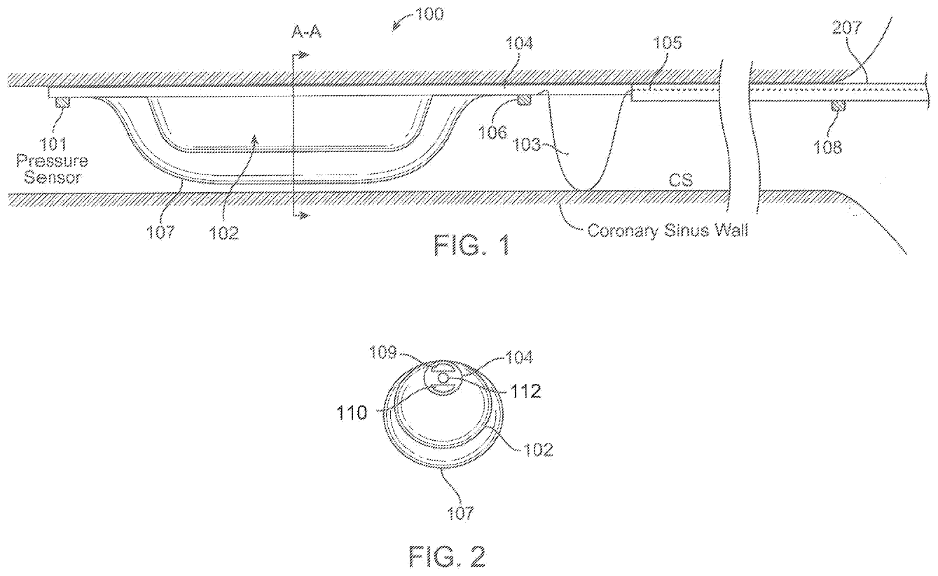

As illustrated in FIGS. 1 and 2, in one embodiment of the present disclosure, vascular pressure monitoring system 100 includes catheter body 104, which may be a triple lumen catheter as shown in FIG. 2, or a double lumen catheter in other embodiments. In the illustrated embodiment, catheter body 104 is provided with two inflatable balloons, inner non-compliant balloon 102 and outer compliant balloon 107. Inflation lumen 110 communicates with non-compliant balloon 102 and inflation lumen 109 communicates with outer compliant balloon 107. Central guidewire lumen 112 provides a pathway for a guidewire to facilitate placement. In some embodiments, a single balloon, compliant or non-compliant, may be used. Each lumen of catheter body 104 is used to inflate and deflate balloons 102 and 107 in a conventional balloon catheter manner. In this embodiment, catheter body 104 and balloons 102, 107 are configured to be eccentrically positioned within the vascular lumen in which they are deployed.

The coronary sinus (CS) is depicted in FIG. 1, but other vascular lumens may be monitored. Anchor wire 103 is configured to temporarily fix the distal end of catheter body 104 and balloons 102, 107 at a monitoring site and to maintain them pushed up against the lumen wall at one side of the vascular lumen. Anchor wire 103 is deployed and retracted via control wire 105, which may also traverse guidewire lumen 112. Catheter body 104 and anchor wire 103 are delivered and retrieved from a monitoring site in the vascular lumen via guide catheter 207. Multiple pressure sensors may be included in system 100. In the disclosed embodiment of FIG. 1, three pressure sensors are provided: pressure sensor 101 at the distal end of catheter body 104, pressure sensor 106 at the proximal sides of balloons 102, 107 and, more distally on guide catheter 207, pressure sensor 108 is disposed to allow for more proximal sensor measurements, such as in the right atrium (RA) when system 100 is deployed in the coronary sinus (CS).

With the embodiment shown in FIG. 1, inner, non-compliant balloon 102 provides a fixed-sized vascular occlusion or restriction when inflated. Outer compliant balloon 107 can be inflated at varying sizes to increase the vascular occlusion up to a full occlusion, depending on the type of pressure measurements to be taken. Additional overlapping balloons may be provided.

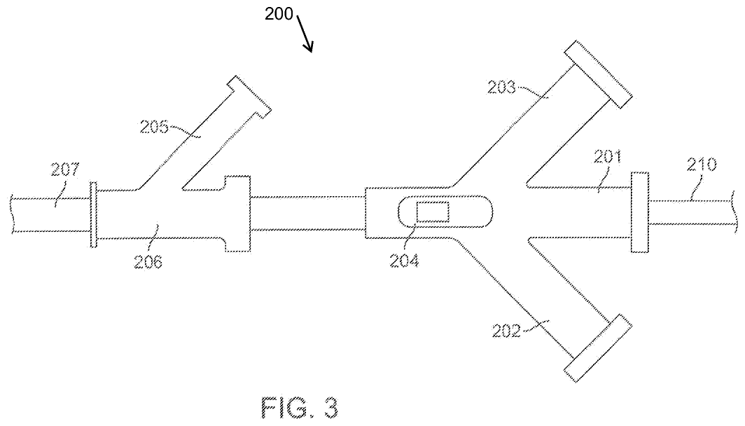

FIG. 3 shows one embodiment of a control hub system 200 for a vascular pressure monitoring system as disclosed herein. As shown therein, distal hub 206 provides for control of guide catheter 207. Port 205 may be configured as an additional inflation port to control inflation and deflation of an additional balloon (not shown) outside of guide catheter 207, which can be inflated to obtain wedge pressure in the coronary sinus. Proximal hub 201 controls catheter body 104 (FIG. 1) and includes inflation ports 202 and 203 for controlling inflation and deflation of balloons 102, 107 via the dual lumens of the catheter body 104. Proximal hub 201 also provides a central lumen port to receive guidewire 210 to facilitate navigation of guide catheter 207 to the vascular monitoring site of interest. With guide wire 210 removed, the central lumen port may be used as a further pressure monitoring port. Control mechanism 204 controls deployment and retraction of anchor wire 103 via control wire 105 (FIG. 1).

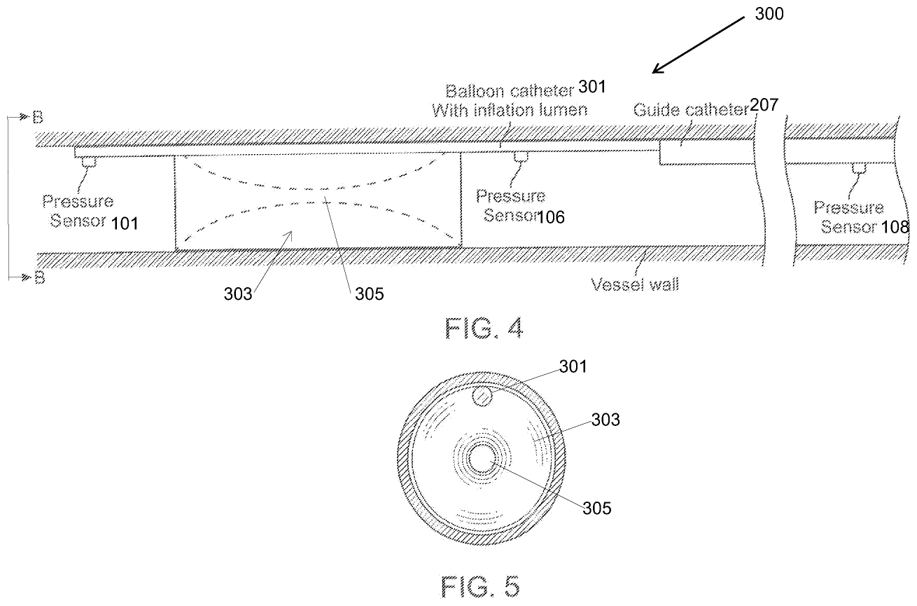

An alternative vascular pressure monitoring system 300 is shown in FIGS. 4 and 5. In this embodiment, a catheter body 301 is delivered through a guide catheter 207 to the vascular lumen monitoring site, which may in some embodiments be the coronary sinus. A balloon 303 configured to provide an internal hour-glass shaped orifice 305 is provided in the distal region of the catheter body 301. Inner walls 307 of balloon 303 that form orifice 305 may be constructed of a compliant material that allows the size of orifice 305 to be controlled by inflation pressure thus providing a variable size flow restriction. The balloon 303 may be inflated and deflated through a central inflation lumen in the catheter body, as shown, for example, in FIG. 2. Because the hour-glass shaped balloon 303 is configured to contact the vessel wall around its periphery with the orifice restriction 305 centrally located, a separate anchor wire may not be required in this embodiment. Alternatively, an anchor wire 103, as described hereinabove and shown in FIG. 1, may be included for added anchoring security. Pressure sensors 101, 106 and 108, such as shown and described for the embodiment of FIG. 1 also may be employed in the embodiment of FIG. 4.

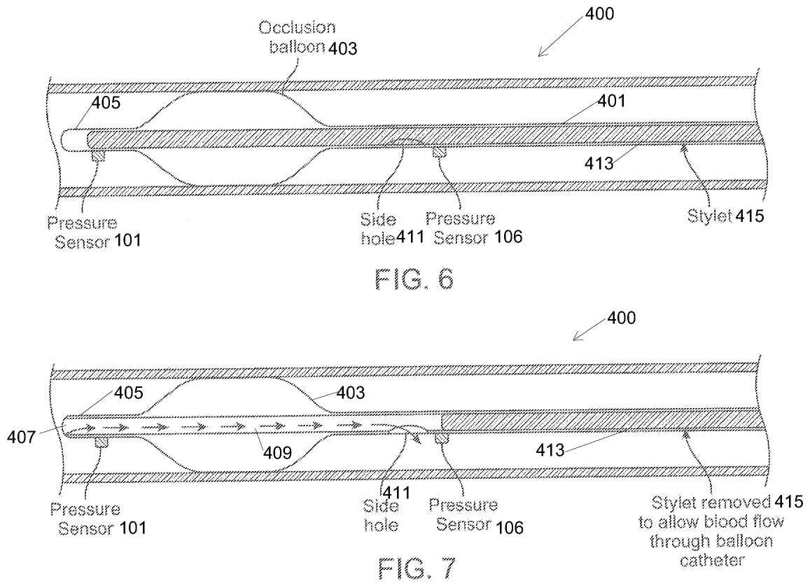

In a further alternative embodiment 400, shown in FIGS. 6 and 7, a catheter body 401 includes a distally positioned occlusion balloon 403. An inflation lumen (not shown) for inflating the balloon is provided in the catheter body. At the distal tip 405 of the catheter body, distal to the occlusion balloon 403, a central blood flow port 407 is provided and defines the entry to a reduced orifice diameter flow passage 409. Side hole 411 proximally disposed with respect to the occlusion balloon 403 provides the exit port for the reduced orifice flow passage. The catheter body 401 also defines a central lumen 413 for a retractable stylus 415 that can be used to allow access to or close the reduced orifice diameter flow passage 409. With the stylus 415 fully extended distally, the entry port 407 and flow passage 409 is blocked entirely. Retracting the stylus distal end to a position proximal of the side hole 411 allows maximum flow through the reduced orifice diameter flow passage 409. Alternatively, the position of the stylus 415 may be advanced somewhat distally from the fully open position shown in FIG. 7 so as to further reduce flow through the flow passage. As in other embodiments, distal and balloon proximal pressure sensors 101, 106 are included. A further proximal pressure sensor, positioned for example as pressure sensor 108 in FIG. 1 may be employed.

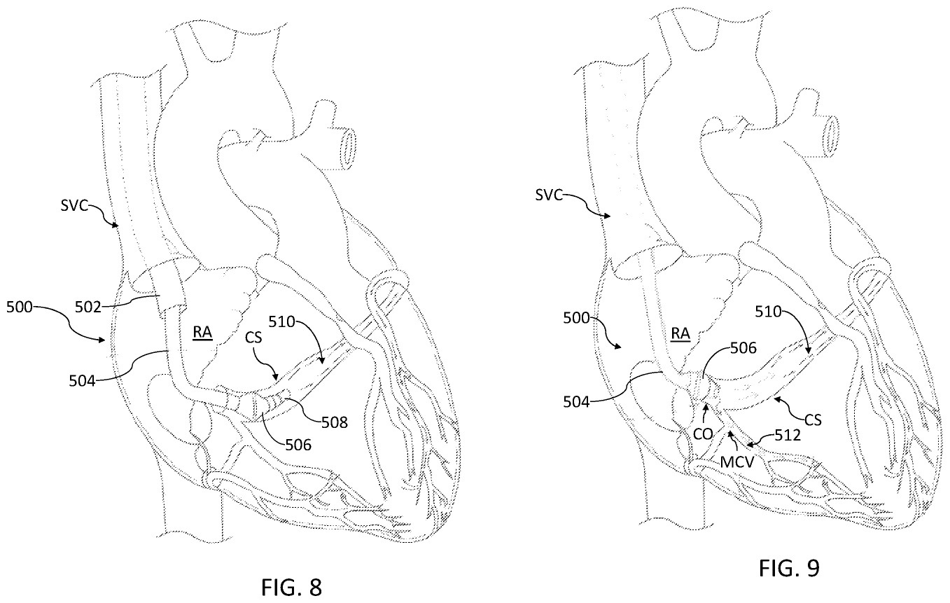

As illustrated in FIGS. 8 and 9, a system 500 generally comprises guide catheter 502 introduced via the superior vena cava (SVC); flow occlusion catheter 504 with distally configured occlusion device 506 and distal end port 508 positioned in the coronary sinus (CS). As will be appreciated by persons of ordinary skill based on the teachings of the present disclosure, system 500 may, in general, comprise any combination of the alternative embodiments of pressure monitoring systems, catheters and occlusion devices herein disclosed. For example, occlusion catheter 504 may alternatively comprise any of catheter body 104, 301, 401 and other alternatives described herein and occlusion device 506 may comprise any of balloons 102/107, 303, 403 or other occlusion devices as well as variations thereof as further described herein.

In one example of use, system 500 can be used to infuse therapeutic agents 510 retrograde up the coronary sinus (CS) to improve/optimize myocardial salvage and reperfusion to limit the infarct size. FIG. 9 shows the retrograde infusion 512 into the middle cardiac vein (MCV) that would enable the inferior wall (RCA STEMI patients) to be treated. The occlusion device 506 can be placed in at the coronary ostium (CO) to prevent flow into the right atrium (RA) and allow specific infusion to the venous system to provide direct reperfusion retrograde to optimize myocardial tissue.

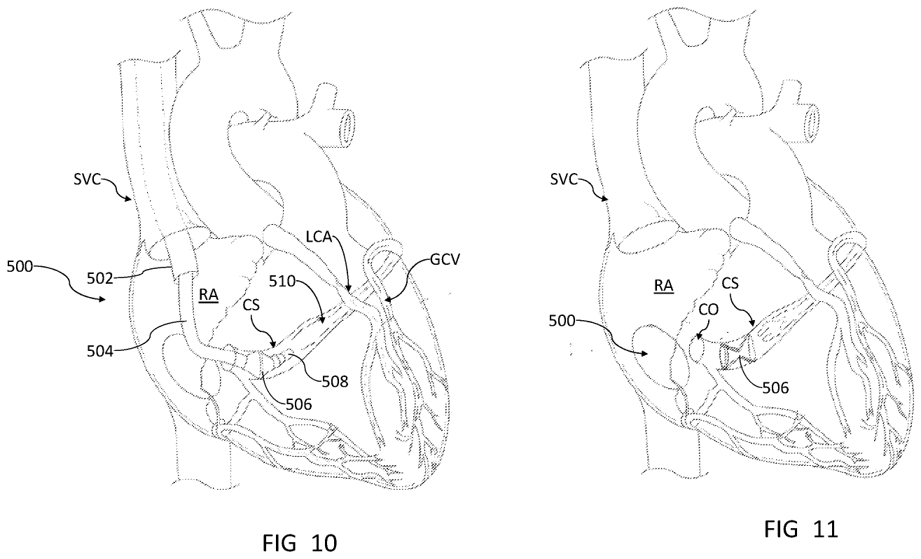

It is also advantageous to inject contrast within the vasculature while the coronary sinus is occluded. The injection of contrast will remain longer within the vasculature and could give valuable information about the retrograde flow within the heart vessels as depicted in FIG. 10. Additional valuable information may be obtained regarding the redistribution of blood flow into the ischemic areas of the heart during intermittent occlusion and/or retrograde reperfusion.

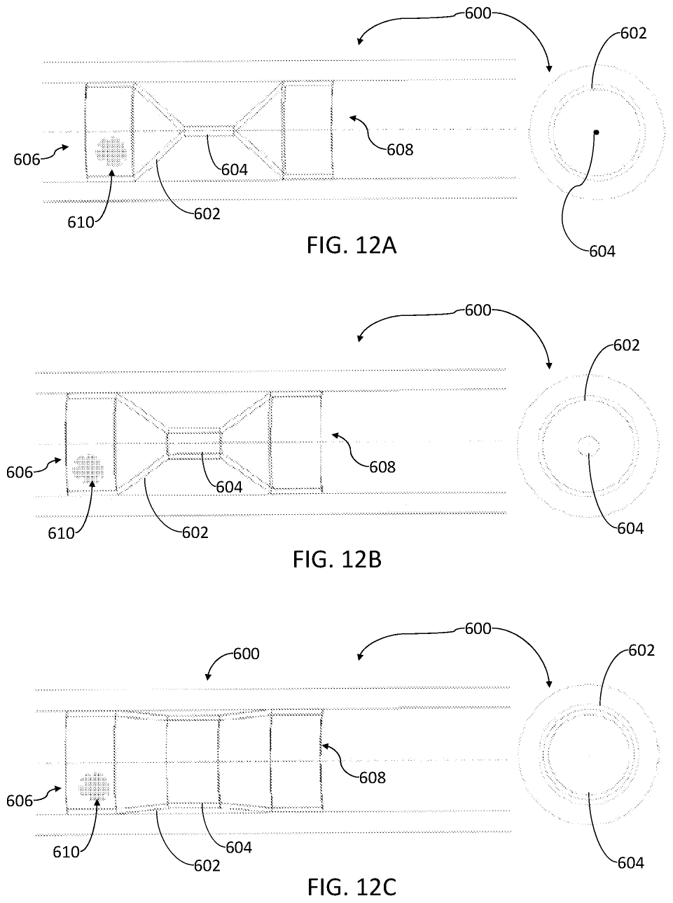

As illustrated in FIG. 11, in some embodiments, occlusion device 506 would not be permanently tethered to catheter 504 and would be an implant (such as one of implants 600, 650, 700, 750, etc. described below) that can be positioned within the coronary sinus (CS). FIG. 11 schematically depicts implant 506 as an hourglass-shaped implant of the types shown in FIGS. 12A-C and 15A-C. However, other configurations of flow-restricting implants as described herein also may be employed and placed to impede flow and cause retrograde perfusion in the vessel.

Unlike prior flow restricting devices, embodiments described herein are configured to optionally completely block flow within the coronary sinus to provide a spike retrograde pressure. For example, occlusion device 600 as shown in FIGS. 12A-C may be configured with an hourglass-shaped body 602 having an adjustable orifice restriction 604 flanked by flow entry end 606 and flow exit end 608. Hourglass-shaped body 602 may be from an expandable metal construct 610 comprising a woven wire mesh or laser cut tube structure with or without a coating layer. The occlusion provided by adjustable orifice restriction 604 can be fully closed as shown in FIG. 12A, opened thereafter and cycled as shown in FIG. 12B in a flow restricting state, and fully opened as shown in FIG. 12C employing various actuation means based on the teachings of the present disclosure.

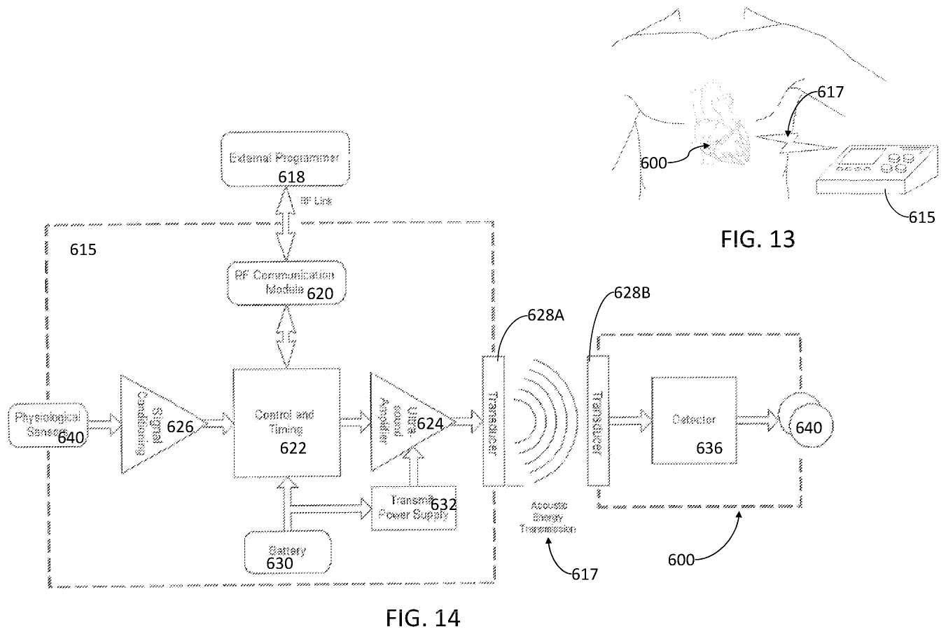

FIG. 13 shows utilizing a communication/power link 617, for example RF energy or ultrasound energy, outside the patient body to control the opening and closing of a device implanted into a patient's coronary sinus. In that way, the patient would not need to be connected to a catheter and within the Cath lab for hours. The device could be retrieved after 24 hours or longer depending on the perfusion of the vessels within the heart. FIG. 14 shows the RF communication and ability to control the implant remotely utilizing acoustic energy. In one example, as shown in FIG. 14, control system 615 includes control and timing circuit or processor 622, which communicates with physiological sensors 650 (e.g. pressure sensors 101, 106) via signal conditioner 626, which may provide functions of signal conversion, linearization, amplification, and filtering among other functions as is understood by persons skilled in the art. Circuit or processor 622 provides outputs to ultrasound amplifier 624, which in turn drives transducer 628A. Power is supplied via battery 630 and power supply 632. A user may communicate with control system 615 via external programmer 618 and RF communications module 620. In such an embodiment, occlusion device 600 (representing generically any of the occlusion device embodiments disclosed herein) may include transducer 628B receiving signals from transducer 628A, detector processor or circuit 636 and occluder actuator 640 as described in connection with specific embodiments hereinbelow.

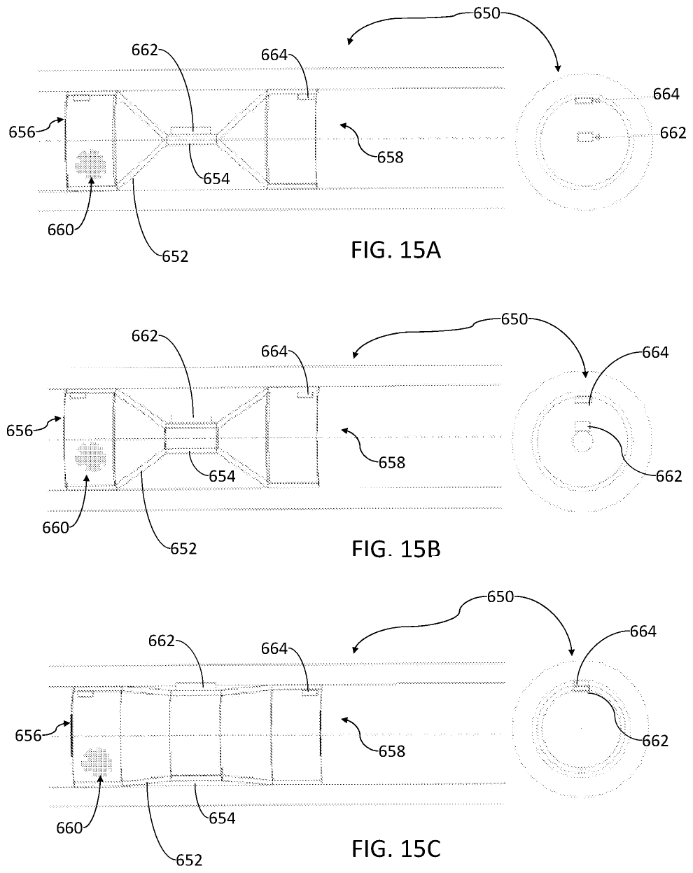

A further alternative embodiment of a remotely controllable occlusion device 650 is shown in FIGS. 15A-C. As depicted therein, occlusion device 650 has an hourglass-shaped body 652 having an adjustable orifice restriction 654 flanked by flow entry end 656 and flow exit end 658. Hourglass-shaped body 652 may be from an expandable metal construct 660 comprising a woven wire mesh or laser cut tube structure with or without a coating layer. A pressure sensor 664 attached near the orifice 654 as shown in FIG. 15A, where the orifice 654 is in a closed state occluding the vessel. There is a means of actuation 662 responsive to the sensed pressure state via sensor 664 to change the orifice diameter using mechanical, electromechanical and/or acoustics that cause vibration of the orifice 654 within the implant. In one example, actuation means may comprise power remotely delivered via RF and received by the implant. The power results in resistive heating of nitinol shape memory wire with an Austenitic start temperature configured to be above body temperature, but below a safe operating temperature (e.g. 60° C.). Austenitic transformation of the Nitinol causes the shape of the hourglass to change states from either open to closed or vice versa. When the energy is removed, the Nitinol will again be come martensitic and thus ductile allowing the original shape of the hourglass to return. The pressure can be monitored, and the orifice adjusted to a restrictive state allowing partial flow through the vessel as shown in FIG. 15B. The actuator means 662 can open the restriction completely allowing for full blood flow back through the vessel depicted in FIG. 15C.

Another embodiment 700 can utilize a turbine 702 that can rotate within a cylinder 704 under motive force provided blood flow across turbine blades 710. The two plates have partial openings 706/708 that when rotated can occlude flow, allow for partial flow, or open flow. FIG. 16A shows an expanded view of the implant 700 with the turbine 702 outside the outer cylinder 704. FIG. 16B shows the device 700 as implanted. FIG. 16C shows the two apertures 706/708 preventing flow through since the apertures are completely overlapping, whereas FIG. 16D has partial flow since the two apertures 706/708 are only partly overlapping. FIG. 16E the two apertures 706/708 are aligned to allow flow through the opening. This embodiment is intermittent without the need for a tether or catheter. The rate of intermittent rotation is controlled by the blood flow passing through it and the ‘pitch’ of the turbine blades 710.

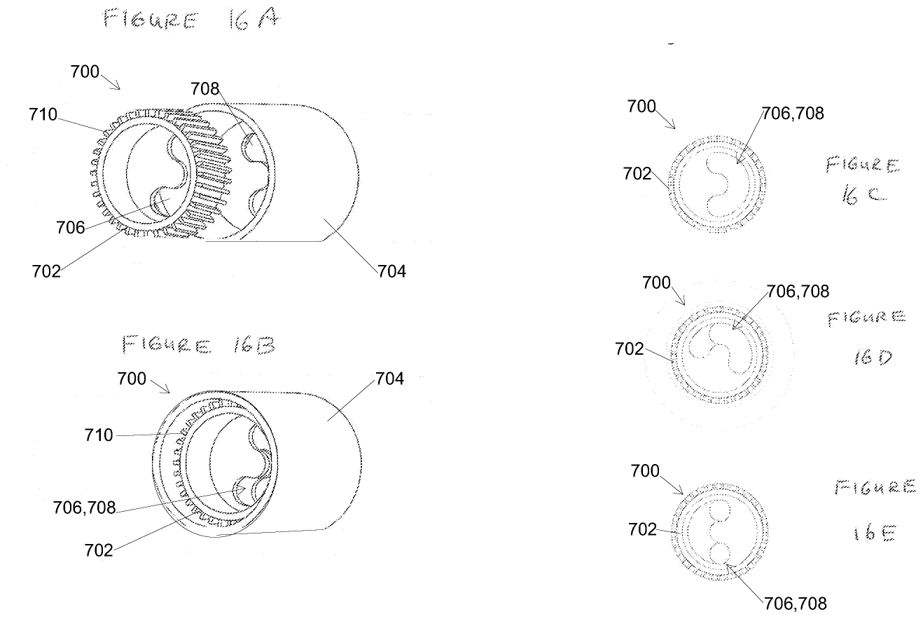

In a further alternative embodiment, in occlusion device 750 the apertures 756/758 can be rotated using electromagnetic energy source see FIGS. 17A and 17B. Externally applied and controlled electromagnetic energy can be oscillated to cause movement of fixed pole magnets on the implant resulting in a rotation or other movement. The oscillation can be accomplished with either a battery source or with an external energy source. The apertures rotation is shown in FIGS. 17C and 17D.

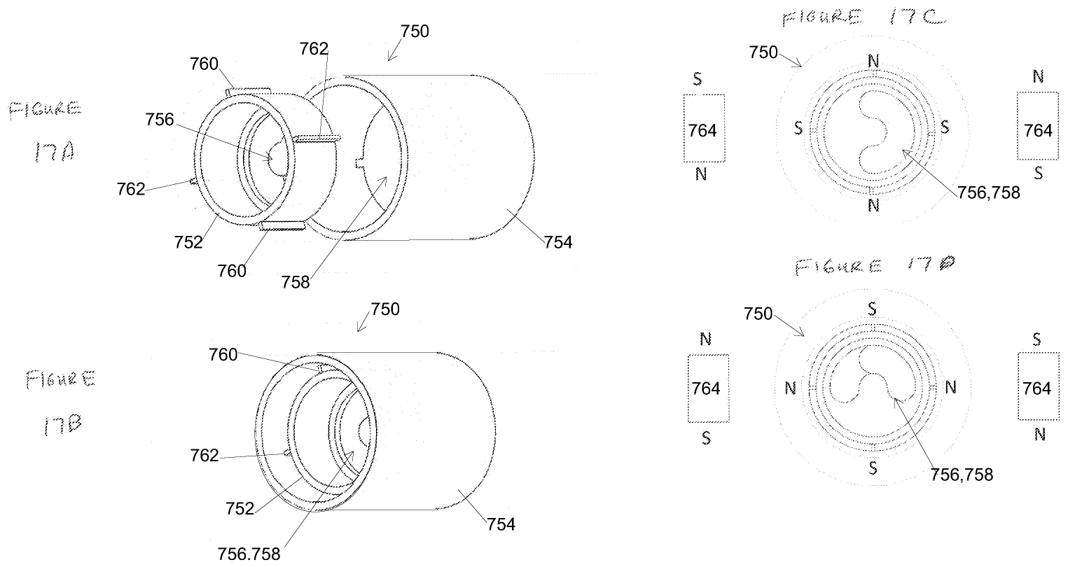

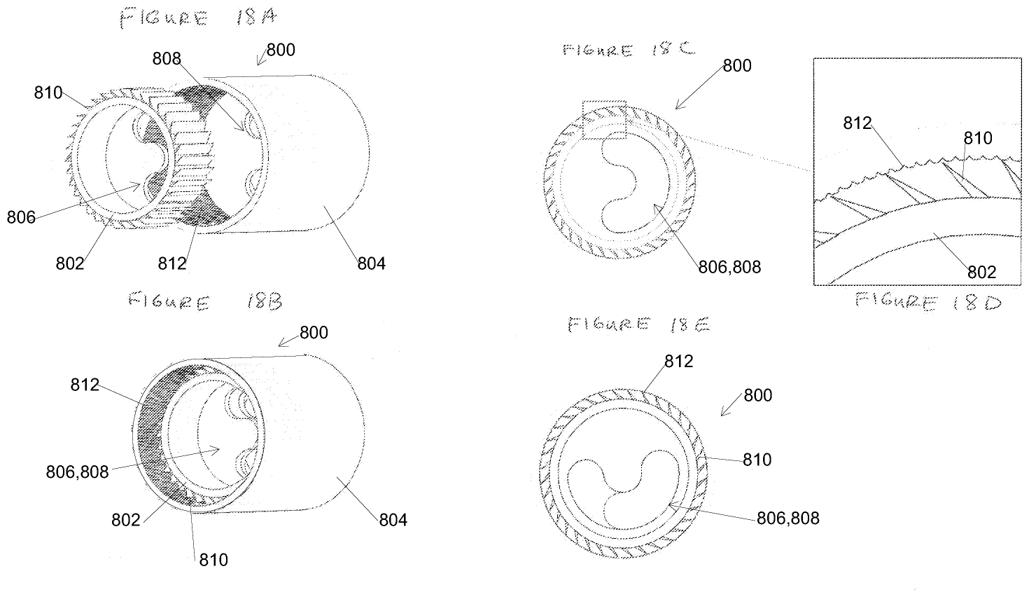

In another alternative embodiment, occlusion device 800 may use other energy sources that can rotate the apertures in ultrasound or directional vibration as shown in FIGS. 18A-E. As shown therein, inner turbine 802 has outwardly extending fan blades 810 that engage a saw-toothed surface 812 around the inner periphery of cylindrical housing 804 forming a vibrationally controlled ratcheting mechanism. Vibration of a ratcheting mechanism can cause movement in a controlled direction. Random movement skips a gear and advances the ratchet which prevents it from moving backwards. A fine tooth ratcheting mechanism around the perimeter of the implant would allow a directional, rotational movement to result. Elongate apertures 806/808 are provided in plates across the center of each part and can be aligned by rotation to control the size of the opening. FIG. 18D is an exploded view of the fan blades 810 that can be rotated with this acoustic energy source by vibration against sawtooth surface 812. The implant can be designed to allow for the blood pressure itself to pulsate and rotate the fan blades rotating the aperture within the implant.

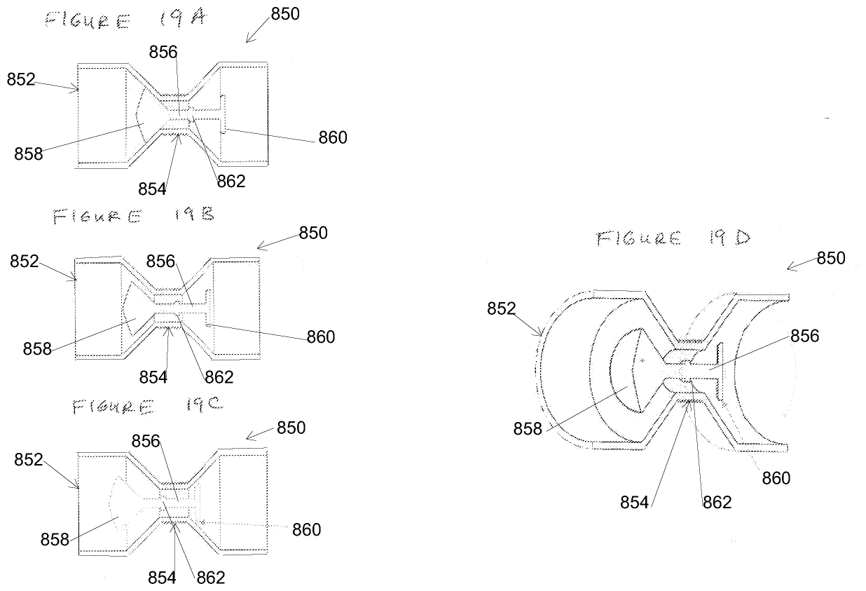

Another embodiment is an hourglass occlusion device 850, having an hourglass-shaped body 852 with a conical piston 858 formed on a remotely actuatable plunger 856 that extends across adjustable orifice 854 as shown in FIG. 19A. The piston or cone can be RF coupled to induce an electromagnetic motion to close against the opening in the hourglass restrictor. Central magnet 862 is a fixed pole magnet that is translated back and forth by the changes in the electromagnetic force applied. Retaining bar 860 prevents inadvertent removal of plunger 856. The piston/cone can be displaced linearly to allow for partial flow around the cone as shown in FIG. 19B and the normal patient blood flow could provide enough pressure to open the cone in a fail-safe mode to ensure that the implant does not stay occluded if power is lost shown in FIG. 19C (open). FIG. 19D is an isometric view to show that the arms do not cause the device to impeded flow whereas the cone when moved into the hourglass implant would cause occlusion.

The occlusion devices in FIGS. 12A-C, 15A-C, 16A-E, 17A-D, 18A-E and 19A-D can be removed from the body at a predetermined time to open the vessel to its normal size. Alternatively, the devices may be constructed with bioabsorbable material that would dissipate over a known time period leaving only the cylinder behind. In one example, the bioabsorbable components can be manufactured using a salt that would last only a few hours.

The foregoing has been a detailed description of illustrative embodiments of the disclosure. It is noted that in the present specification and claims appended hereto, conjunctive language such as is used in the phrases “at least one of X, Y and Z” and “one or more of X, Y, and Z,” unless specifically stated or indicated otherwise, shall be taken to mean that each item in the conjunctive list can be present in any number exclusive of every other item in the list or in any number in combination with any or all other item(s) in the conjunctive list, each of which may also be present in any number. Applying this general rule, the conjunctive phrases in the foregoing examples in which the conjunctive list consists of X, Y, and Z shall each encompass: one or more of X; one or more of Y; one or more of Z; one or more of X and one or more of Y; one or more of Y and one or more of Z; one or more of X and one or more of Z; and one or more of X, one or more of Y and one or more of Z.

Various modifications and additions can be made without departing from the spirit and scope of this disclosure. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present disclosure. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this disclosure.

Exemplary embodiments have been disclosed above and illustrated in the accompanying drawings. It will be understood by those skilled in the art that various changes, omissions and additions may be made to that which is specifically disclosed herein without departing from the spirit and scope of the present disclosure.

1. A vascular pressure monitoring system, comprising:
a catheter body defining at least a guidewire lumen and an inflation lumen;
a balloon communicating with the inflation lumen and distally disposed on the catheter body to define a vascular flow restriction when inflated within a vascular lumen;
a distal pressure sensor disposed distally with respect to the balloon; and
a proximal pressure sensor disposed proximally with respect to the balloon.

2. The vascular pressure monitoring system of claim 1, wherein said balloon defines a variable flow restriction.

3. The vascular pressure monitoring system of claim 1,
further comprising:
a guide catheter configured to receive said catheter body; and
a second proximal pressure sensor disposed on said guide catheter.

4. The vascular pressure monitoring system of claim 1, wherein said balloon comprises:
an inner, non-compliant balloon; and
an outer compliant balloon.

5. The vascular pressure monitoring system of claim 4, wherein the catheter body comprises a triple lumen catheter with a first inflation lumen communicating with the inner, non-compliant balloon, a second inflation lumen communicating with the outer compliant balloon and a guidewire lumen.

6. The vascular pressure monitoring system of claim 4,
wherein:
said inner and outer balloons are eccentrically disposed on the catheter body; and
said pressure monitoring system further comprises retractable anchor wire on the catheter body configured to position the catheter body against the vascular lumen wall at a pressure monitoring site.

7. The vascular pressure monitoring system of claim 1, wherein
said balloon comprises an hour-glass shaped balloon defining an internal blood flow orifice.

8. The vascular pressure monitoring system of claim 7, wherein said internal blood flow orifice is defined by an inner, annular compliant balloon wall to provide a variable flow restriction based on degree of inflation of said balloon.

9. The vascular pressure monitoring system of claim 1, wherein:
said balloon is disposed around said catheter body; and
the catheter body defines flow passage through the balloon having an open distal end defining one blood flow port and a side hole proximal to the balloon defining a second blood flow port.

10. The vascular pressure monitoring system of claim 9, further comprising a flow control stylus disposed in said flow passage.

11. The vascular pressure monitoring system of claim 1,
wherein the catheter body is further configured to deliver therapeutic or diagnostic agents into the vasculature across the flow restriction.

12. An implantable variable vascular flow restriction device, comprising:
an implantable body member configured to be implanted in a vascular lumen and to extend across the vascular lumen fully around the inner periphery of the vascular lumen;
variable flow restricting means disposed within said implantable body member; and
means for controlling the variable flow restricting means.

13. The implantable vascular flow restriction device of claim 12, wherein said variable flow restricting means comprises:
a pressure sensor configured to produce a local vascular pressure signal; and
an adjustable diameter orifice responsive to said pressure signal.

14. The implantable vascular flow restriction device of claim 13, wherein said means for controlling further comprises a vibratory energy source cooperating with the variable flow restricting means to set the adjustable orifice diameter in response to said pressure signal.

15. The implantable vascular flow restriction device of claim 13, wherein said means for controlling further comprises:
a remotely deliverable heating source responsive to said pressure signal; and
a shape-memory alloy having a temperature variable dimensional property forming said adjustable diameter orifice, whereby delivery of heat alters the orifice diameter and removal of heat returns the orifice diameter to an original size.

16. The implantable vascular flow restriction device of claim 15, wherein said shape-memory alloy is Nitinol and said heating source is RF energy.

17. The implantable vascular flow restriction device of claim 12, wherein said variable flow restricting means comprises:
a fixed plate defining a first elongate opening; and
a rotatable plate defining a second elongate opening wherein the second plate overlies the first plate and rotation of the second plate relative to the first plate aligns said elongate openings to provide a variable area flow passage.

18. The implantable vascular flow restriction device of claim 17, wherein the rotatable plate is fixed within a rotatable turbine disposed within said implantable body member.

19. The implantable vascular flow restriction device of claim 18, wherein said means for controlling comprises radially projecting turbine blades disposed around an outside periphery of said rotatable turbine such that blood flow across said turbine blades rotates the rotatable plate relative to the fixed plate to alter the flow passage area.

20. The implantable flow restriction device of claim 18, wherein the turbine includes a plurality of radial magnetic elements and said means for controlling comprises external electro-magnets.

21. The implantable flow restriction device of claim 18, wherein:
an inner peripheral surface of the implantable body member is formed with saw-tooth protrusions;
an outer peripheral surface of the rotatable turbine is formed with ratchet teeth engaged with said saw-tooth protrusions; and
said means for controlling comprises an external ultrasonic actuator configured to vibrate the rotatable turbine relative to the implanted body member whereby said vibrations advance said ratchet teeth against the saw-tooth surface.

22. The implantable vascular flow restriction device of claim 12, wherein said variable flow restricting means comprises a variably positional flow control plunger disposed in a fixed diameter orifice.

23. The implantable vascular flow restriction device of claim 22, wherein:
said flow control plunger includes a centrally disposed magnetic member; and
said means for controlling comprises an externally applied magnetic field.

24. A vascular diagnostic or treatment method, comprising:
placing a vascular pressure monitoring catheter into a vessel lumen at treatment or monitoring site;
inflating a distal balloon on said catheter at said treatment or monitoring site to at least partially occlude the vessel lumen;
delivering a therapeutic or diagnostic agent into the vessel lumen through said catheter beyond the partial occlusion; and
monitoring patient response to said therapeutic or diagnostic agent.

25. The method of claim 24, further comprising altering the partial occlusion of the vessel lumen by changing size or shape of said balloon in response to pressure monitored distally and proximally with respect to said balloon.

26. The method of claim 25 wherein said altering the partial occlusion of the vessel lumen comprises temporary full occlusion.

27. The method of claim 24, wherein said placing comprises positioning said distal balloon in the patient's coronary sinus upstream of the middle cardiac vein and said delivering comprises retrograde infusion of the therapeutic or diagnostic agent into the coronary sinus.

28. The method of claim 24, wherein said placing comprises positioning said distal balloon in the coronary sinus downstream of the middle cardiac vein and said delivering comprises retrograde infusion of the therapeutic or diagnostic agent into the middle cardiac vein.

29. The vascular pressure monitoring system of claim 4, wherein said balloon defines a variable flow restriction
